(19) United States
(12) Patent Application Publication
(10) Pub. No.: US 2026/0325310 A1
(43) Pub. Date: Oct. 22, 2026

(54) REVISION FEMUR PROSTHESIS

(71) Applicant: BIORAD MEDISYS PRIVATE LIMITED, Pune (IN)

(72) Inventors: Debasish PRADHAN, Pune (IN); Ankita PATIL, Pune (IN)

(21) Appl. No.: 19/109,578

(22) PCT Filed: Sep. 8, 2023
(86) PCT No.: PCT/IN2023/050852

§ 371 Date: Mar. 7, 2025

(30) Foreign Application Priority Data
Sep. 8, 2022 (IN) 202221051297

(51) Int. Cl. A61F 2/38; A61F 2/30

(52) U.S. Cl. CPC A61F 2/3859; A61F 2/30734; A61F 2/389; A61F 2002/3069; A61F 2002/3082; A61F 2310/00029; A61F 2310/00059; A61F 2310/00101

(57) ABSTRACT

A femur prosthesis is disclosed. The femur prosthesis comprising a femoral component. The fabricated from Cobalt-Chromium-Molybdenum configured for attachment to a femur, the femoral component having a 3d curved profile in its box 202. Further the femur prosthesis comprising a tibial insert. The tibial insert is mounted on the femoral component. The tibial insert made of Ultra-High Molecular Weight Polyethylene material, wherein the tibial insert comprises an articulating surface. The articulating surface interfaces with the femoral component to facilitate flexion-extension and internal-external rotational movement. The tibial insert comprises a tibial post. The tibial post comprises an increased width in medio-1 lateral and anterior-posterior directions, wherein based on the 3d curved profile in the box, an internal-external rotation progressively increases based on different flexion angles of the femoral component, achieving an internal-external angle ranging between 0.4° to 5.5°.

### TECHNICAL FIELD

The present subject matter relates generally to orthopedic prostheses, and more specifically, to a revision femur prosthesis with progressive internal-external angle change at different flexion angles.

### BACKGROUND

Advances in surgical technique and implant technology have improved the ten-year survival after primary total hip arthroplasty (THA). Despite this, the number of revision procedures has been increasing in recent years, a trend which is predicted to continue. Revision THA is a technically demanding procedure often complicated by a loss of host bone stock which may be compounded by the need to remove primary implants. Both cemented and uncemented implant designs are commonly used in the United Kingdom for primary and revision THA and much controversy still exists as to the ideal method of stem fixation. Revision THA is most often indicated for instability, aseptic loosening, osteolysis, infection, periprosthetic fracture, component malposition, and catastrophic implant failure. Understanding the etiology of THA failure is essential for guiding clinical decision making. Femoral component revision presents a complex challenge to the arthroplasty surgeon because of modern implant design as well as bone loss in the proximal femur. Thorough patient evaluation, defect classification, and well-executed surgical reconstruction based on comprehensive preoperative planning may determine the postoperative results. Knowledge of various reconstructive options and the indications for each is necessary to achieve a successful outcome.

Conventional application such at one illustrated in U.S. Pat. No. 5,147,405 disclosed a femur prosthesis including a tibial component. The patent describes a significant emphasis on controlling the ‘rollback’—the movement of contact points in the posterior direction during knee flexion. While this is controlled to a certain degree, the distance of rollback is stated to be about 6 mm, which could still lead to potential issues or complications, especially when compared to the initial invention where no such rollback is mentioned. The design in U.S. Pat. No. 5,147,405 stipulates that no rollback occurs upon flexion of the femoral and tibial components from about 45° to the flexed position. This limits the natural movement or create an unnatural sensation during the middle-to-late stages of flexion. On the contrary, the initial invention might provide a more continuous or smooth motion during the entire range of movement, given the absence of such limitations. The design, as described in U.S. Pat. No. 5,147,405, has a multifaceted mechanism, including a posterior cam surface, stabilizing post with multiple sections, and an anchoring rod. This could potentially make the implantation procedure more intricate and might lead to a longer surgical time and recovery period. While the patent mentions feature to reduce the likelihood of dislocation between the femoral and tibial components, the very fact that these features are emphasized suggests a higher inherent risk. The earlier described invention might have a more robust or stable design that inherently reduces the risk of such dislocations. The patent suggests that controlling the rollback is crucial to prevent the tibial tray from flexing or rocking, which might lead to its loosening. However, if there are any malfunctions or unexpected movements, the dependency on the tray's stability might lead to complications. Given the addition of stabilizing posts, anchoring rods, and cam surfaces, the prosthesis as per U.S. Pat. No. 5,147,405 might be bulkier than the previously described invention. This could potentially result in a thicker profile that might not be suitable for all patients or might require more extensive bone resection during surgery. With multiple points of articulation and engagement (like the engaging means of the posterior cam surface between certain flexion angles), there might be increased wear and tear over time, reducing the longevity of the implant.

Another conventional application illustrated in US 2011/125275 A1 a prosthetic joint that includes a first implant component for attachment to a first bone and a second implant component for attachment to a second bone, is disclosed. The first implant component has a condylar portion that includes first and second condylar bearing surfaces and similarly, the second implant component has bearing surfaces that receive and are complementary to the first and second condylar bearing surfaces. Each of the first and second condylar bearing surfaces and each of the bearing surfaces of the second implant component has a cross-section in a coronal plane that exhibits two different radii, and a contact point is established between the first and second condylar bearing surfaces and the bearing surfaces of the second implant component. The bearing surfaces of the respective implant components are configured such that varus and valgus rotation of the first implant component relative to the second implant component causes the contact point to move outwardly.

### SUMMARY

Embodiments of the present disclosure present technological improvements as solutions to one or more of the above-mentioned technical problems.

Before the present subject matter relating to a revision femur prosthesis, it is to be understood that this application is not limited to the particular system(s) and methodologies described, as there can be multiple possible embodiments which are not expressly illustrated in the present disclosure. It is also to be understood that the terminology used in the description is for the purpose of describing the implementations or versions or embodiments only and is not intended to limit the scope of the present subject matter.

This summary is provided to introduce aspects related to a revision femur prosthesis. This summary is not intended to identify essential features of the claimed subject matter nor is it intended for use in determining or limiting the scope of the present subject matter.

In one embodiment a femur prosthesis comprising: a femoral component, and a tibial insert, is disclosed. The femoral component the fabricated from Cobalt-Chromium-Molybdenum (CoCrMo Alloy) configured for attachment to a femur, the femoral component comprises a curved profile in its femoral box. Further the tibial insert is mounted on the femoral component. The tibial insert made of Ultra-High Molecular Weight Polyethylene (UHMWPE) material, the tibial insert comprises an articulating surface, the articulating surface interfaces with the femoral component to facilitate flexion-extension and internal-external rotational movement, the tibial insert comprises a tibial post, the tibial post comprises an increased width in medio-l lateral and anterior-posterior directions, based on the curved profile in the femoral box, an internal-external rotation progressively increases based on different flexion angles of the femoral component, achieving an internal-external angle ranging between 0.4° to 5.5°.

In the embodiment, the femoral component has configurations in both Left and Right orientations and has a provision for distal and posterior augment attachment. Further, the femoral component comprises a deeper patella groove. In the embodiment the tibial post includes a 4° draft added from a bottom surface of the tibial post and the tibial insert has a 7° posterior slope on the articulating surface. The tibial insert comprises chamfers at its anterior and posterior sides and the anterior chamfer facilitates easy patella movement, and the posterior chamfer provides space for ligaments.

Further, at a 0° flexion angle, the internal-external rotation is up to 1° due to clearances in the femur box and tibial post. Additionally, for a 90° flexion angle, the internal-external rotation ranges up to 5.5° to 6°.

### BRIEF DESCRIPTION OF THE DRA WINGS

The foregoing detailed description of embodiments is better understood when read in conjunction with the appended drawings. For the purpose of illustrating the disclosure, there is shown in the present document example constructions of the disclosure; however, the disclosure is not limited to the specific system/apparatus or method disclosed in the document and the drawings.

The present disclosure is described in detail with reference to the accompanying figures. In the figures, the left-most digit(s) of a reference number identifies the figure in which the reference number first appears. The same numbers are used throughout the drawings to refer various features of the present subject matter.

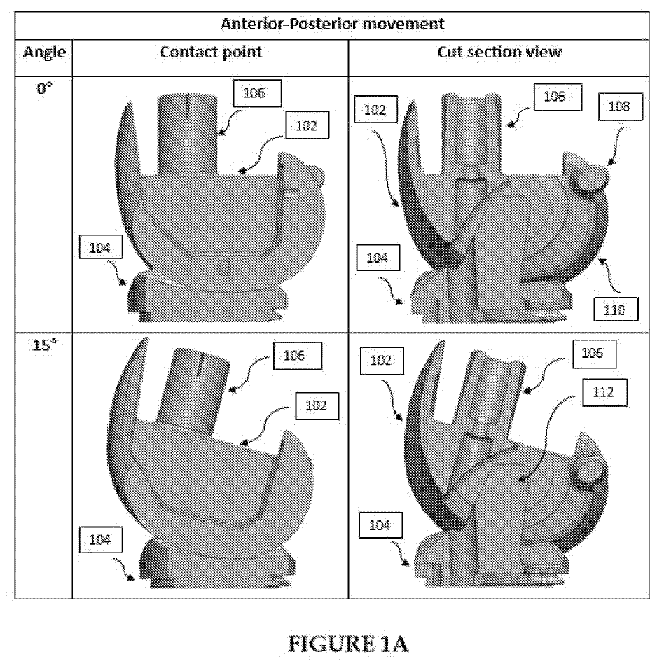

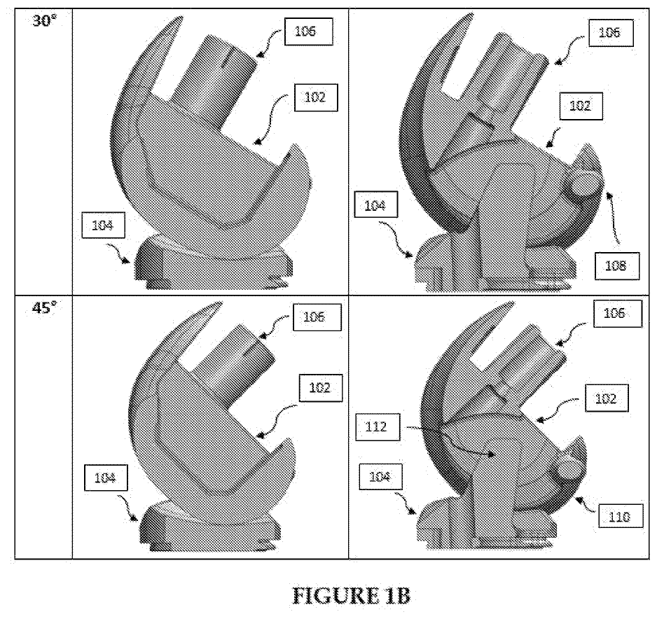

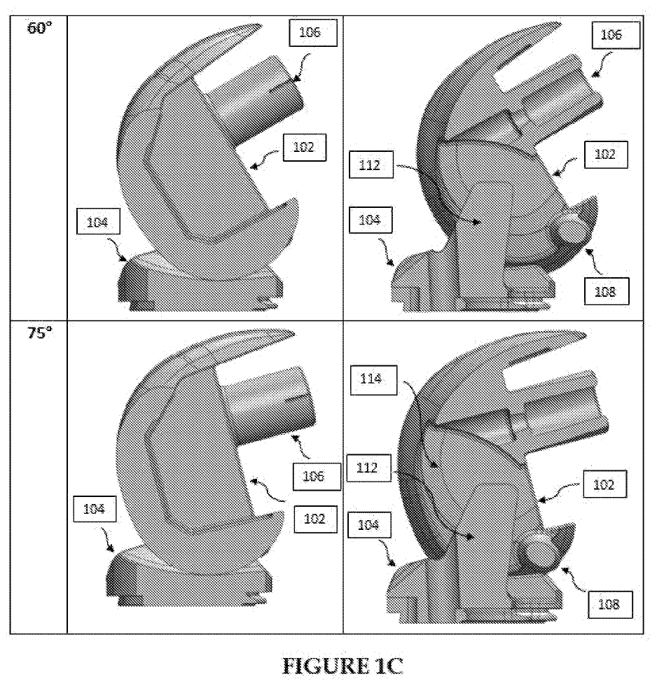

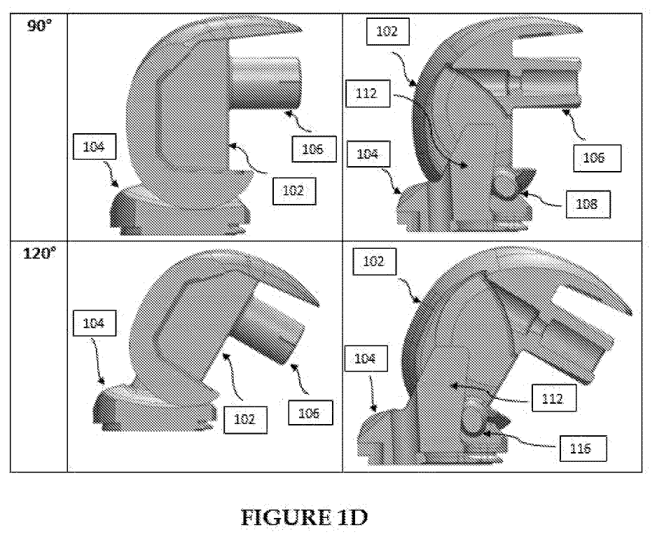

FIG. 1A, FIG. 1B FIG. 1C FIG. 1D illustrates Anterior-Posterior movement, in accordance with an embodiment of the present subject matter.

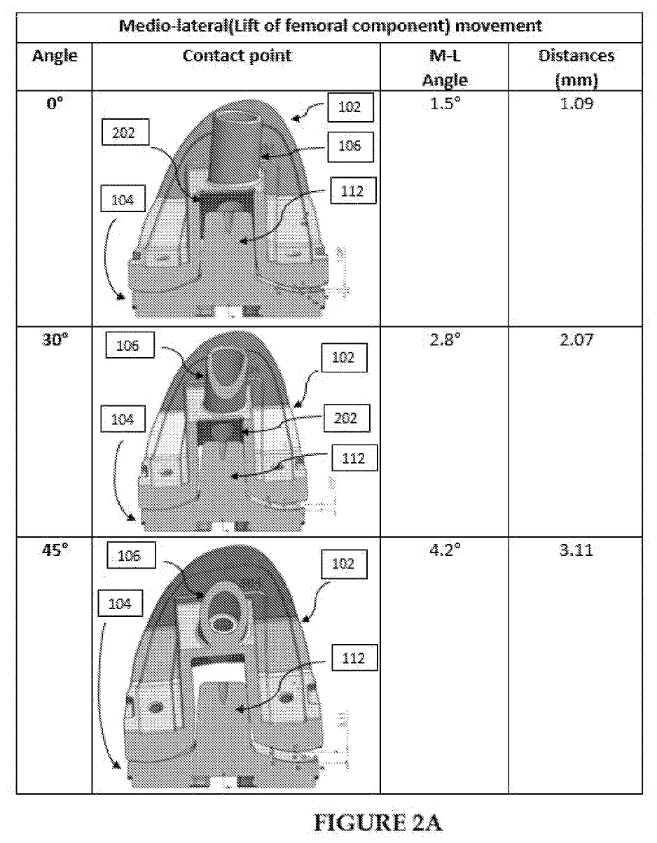

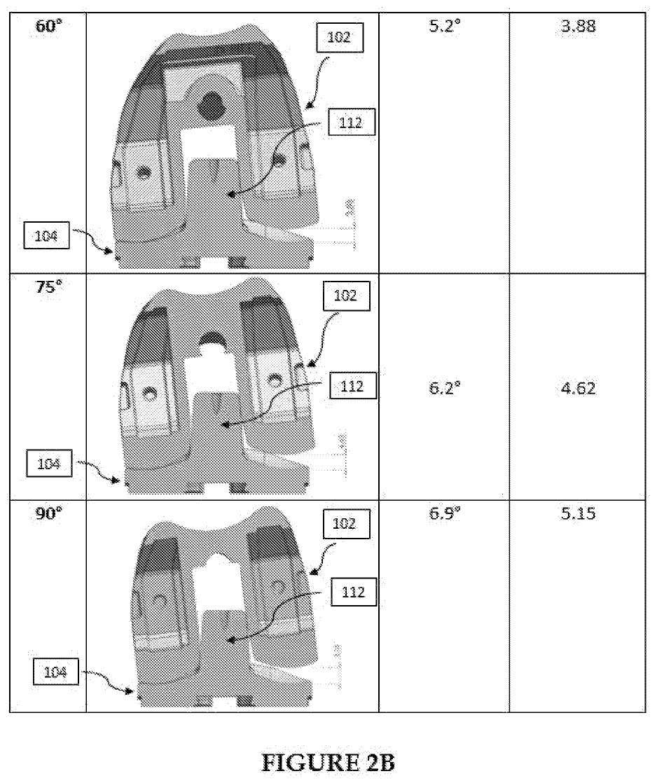

FIG. 2A and FIG. 2B illustrates Medio-lateral (Lift of femoral component) movement, in accordance with an embodiment of the present subject matter.

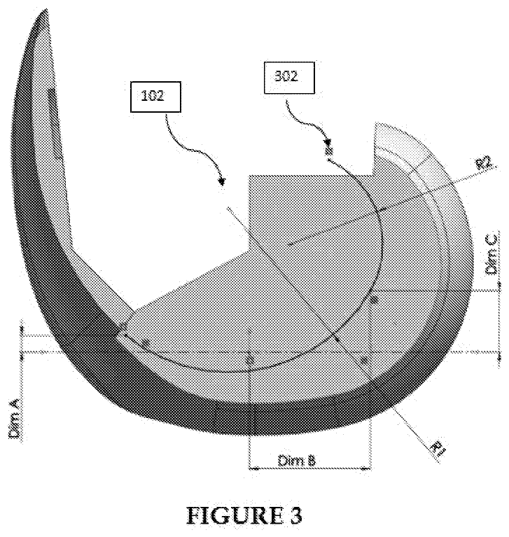

FIG. 3 illustrates, curved dimension of the Femoral components in accordance with an embodiment of the present subject matter.

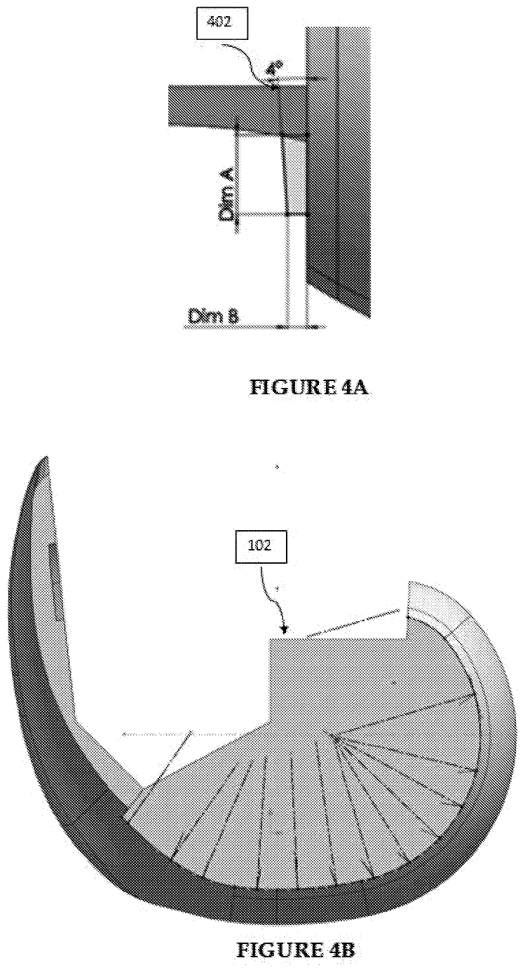

FIG. 4A illustrates Loft profile dimensions with second curve for 3d profile, in accordance with an embodiment of the present subject matter.

FIG. 4B illustrates Section for Loft profile, in accordance with an embodiment of the present subject matter.

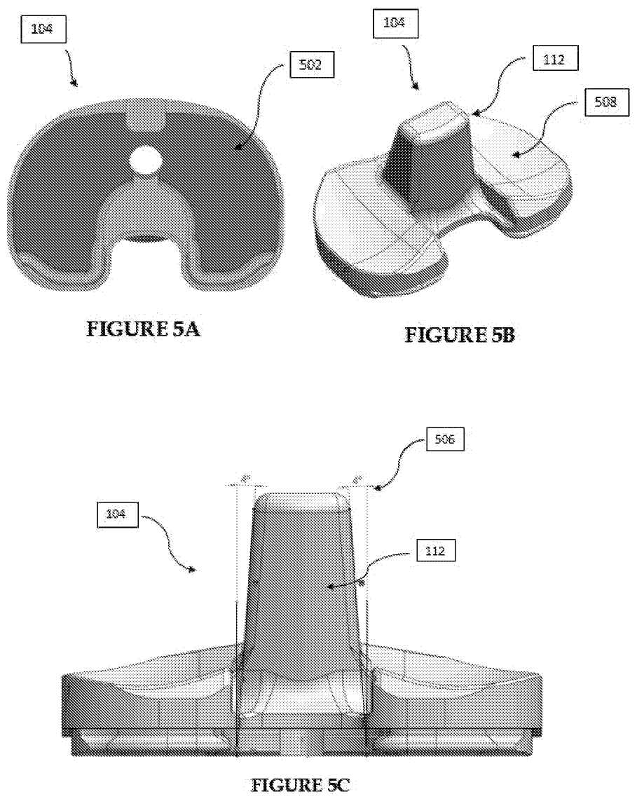

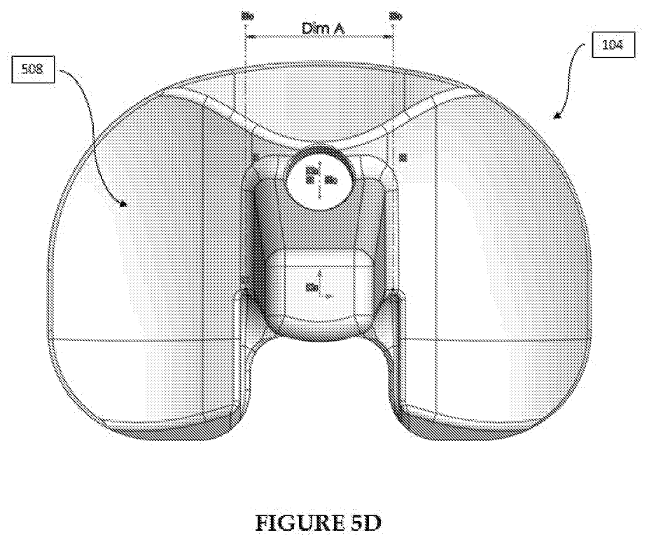

FIGS. 5A, 5B, and 5C illustrates various views of tibial insert, in accordance with an embodiment of the present subject matter.

FIG. 5D illustrates Medio lateral Post distances (Dim A) of tibial insert, in accordance with an embodiment of the present subject matter.

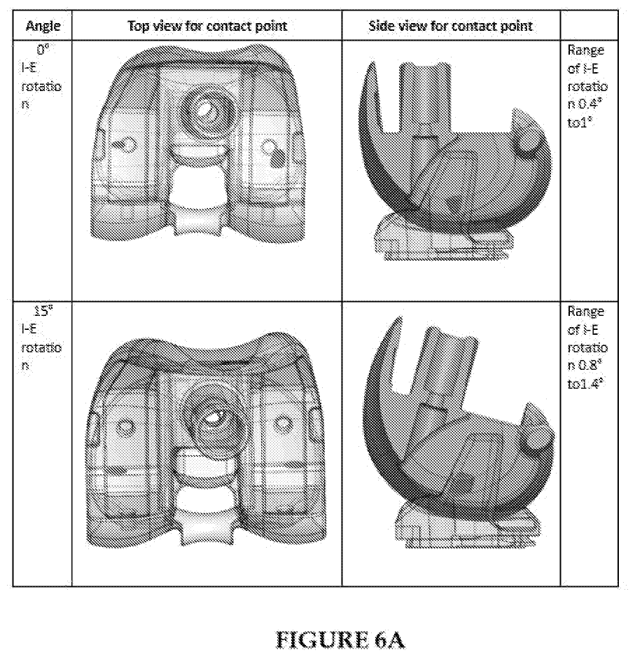

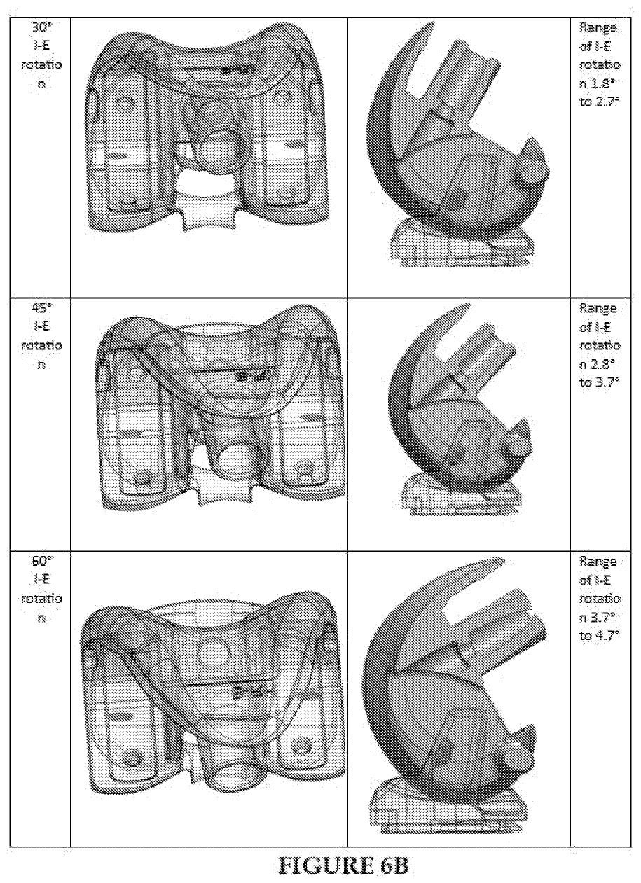

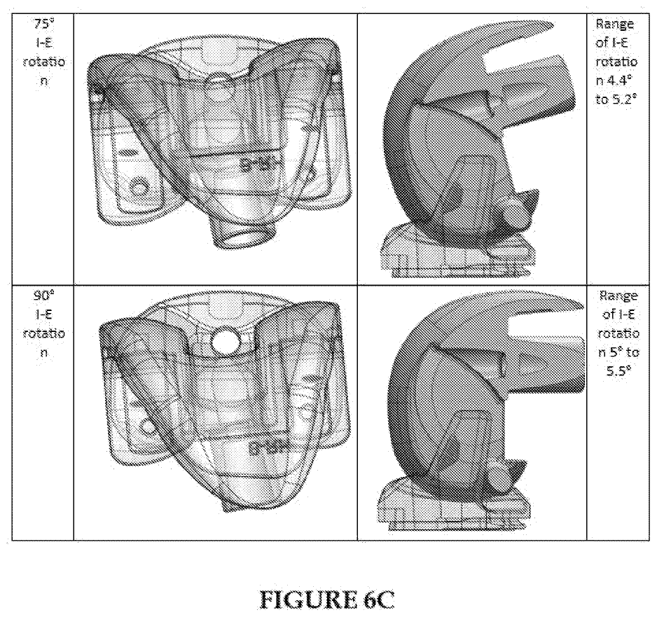

FIG. 6A FIG. 6B and FIG. 6C illustrates Femur insert assembly (after I-E rotation of the femur on the insert contact point are marked), in accordance with an embodiment of the present subject matter.

Further, the figures depict various embodiments of the present subject matter for purposes of illustration only. One skilled in the art will readily recognize from the following discussion that alternative embodiments of the structures and methods illustrated herein may be employed without departing from the principles of the present subject matter described herein.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. Although a revision femur prosthesis, similar or equivalent to those described herein can be used in the practice or testing of embodiments of the present disclosure, the exemplary a revision femur prosthesis is now described.

Various modifications to the embodiment will be readily apparent to those skilled in the art and the generic principles herein may be applied to other embodiments. The present disclosure is not intended to be limited to the embodiments illustrated but is to be accorded the widest scope consistent with the principles and features described herein.

As discussed above, primary total hip arthroplasty (THA) has witnessed a notable improvement in ten-year survival rates due to advancements in surgical techniques and implant technology. However, the number of revision procedures is rising. Femoral component revision presents a complex challenge due to modern implant design and bone loss in the proximal femur. Various femur prostheses have been designed, as exemplified in U.S. Pat. No. 5,147,405 and US 2011/125275 A1. However, there remains a need for a more advanced revision femur prosthesis that addresses the specific technical challenges associated with femoral component revision.

In one embodiment Revision femur-insert progressive angle for revision femur prosthesis, is revision femur prosthesis. Revision femoral component is fabricated from CoCrMo Alloy (Cobalt-Chromium-Molybdenum). Tibial insert is fabricated from Ultra-High Molecular Weight Polyethylene (UHMWPE) material. The articulating surface interfaces with the Tibial Insert to facilitate flexion-extension and internal-external rotational movement. Femur design has both Left and Right configurations with eight different sizes. Femoral components have narrow flange at anterior side to avoid notching with the bone, a complication that could lead to peri-prosthetic fractures. The distal and posterior thicknesses of the femoral component facilitate maintaining adequate joint line, ensuring that the knee's movements post-operation mirror its natural motion as closely as possible. Femoral component having deeper patella groove. It has provision for distal and posterior augment attachment. In revision femoral component 3d curved profile is added for box to facilitate progressive internal-external rotation. Different curve profiles are added in femoral component to get max 5.5° internal-external rotation. Tibial insert are available in eight different sizes with different thicknesses. Tibial inserts have 7° posterior slop on articulating surface. The slope is key for natural biomechanics, ensuring that the knee's movements post-operation mirror its natural motion as closely as possible. Also, the tibial insert has a chamfer at anterior and posterior side. The chamfer at anterior side provides easy patella movement and the chamfer at posterior side provides for easy ligaments movement. Femoral component slide, roll, rotate through tibial insert with different angles with different contact point that is medio-lateral, anterior-posterior movement. There is different contact point at medio-lateral, anterior-posterior movement.

In one embodiment, the present subject matter advantage of progressive internal-external (I-E) angle change based on different flexion angles and includes 3d curve profile in its femoral box (also referred to as box), allowing for a progressive increase in internal-external rotation as the flexion angle changes. Specifically, the rotation can go from up to 1° at 0° flexion 5.5° to 6° at 90° flexion. This not only ensure post-operative patient comfort but also play a pivotal role in enhancing the longevity of the implant, reducing the need for subsequent revisions. Above discussed advantages and other advantages will be better evident in the subsequent section.

In one embodiment Progressive internal-external (I-E) angle change at different flexion angle position related to a revision femur prosthesis, is provided. Further, curved profiles are added in femoral box component. Due to curved profile in femoral box internal-external rotation progressively goes on increasing. At 0° there is slight clearances in femur and insert so there is up-to 1° I-E rotation will occur. When femoral component roll on tibial insert with different flexion angle there is I-E rotation also occur and for 90° flexion angle internal-external angle goes up to 5.5° to 6°. Tibial post width is increased at medio-lateral and anterior-posterior direction to maintain the I-E rotation 0.4° up-to 5.5°. Draft 4° is added on tibial post from bottom surface.

Now referring to the figure, the present subject matter and its embodiments are further elaborated with reference to the figures. Further the below table of nomenclature illustrates the numbers used in the figures.

| Nomenclature | Number |
|---|---|
| Femoral Component | 102 |
| Tibial Insert | 104 |
| Stem Base | 106 |
| Cam | 108 |
| Condyle | 110 |
| Tibial Post | 112 |
| 3D Curve Profile | 114 |
| Cam & Post Alignment | 116 |
| Box | 202 |
| First Profile | 302 |
| Second curve for 3d profile | 402 |
| Reference Plan for Post Angle | 502 |
| Post Angle | 506 |
| articulating surface | 508 |

Referring to FIG. 1A, FIG. 1B FIG. 1C FIG. 1D, Anterior-Posterior movement is disclosed.

Due to clearances in femur box 202 and tibial post 112 there is medio-lateral (Lift of femoral component) movement will create. Further in FIG. 2A and FIG. 2B medio-lateral angle is disclosed. Contact point for femur insert is same for at 15° to 90° angle it will changes when cam 108 engage with tibial post 112. When femur will move in anterior-posterior direction along this internal-external rotation will create. Due to 3d curve profile 114 in femoral box 202 internal-external rotation progressively goes on increasing. At 0° there is slight clarences in femur and insert so there is up-to 1° I-E rotation will occur. When femoral component 102 roll on tibial insert 104 with different angle there is I-E rotation also occur and for 90° flexion angle internal-external angle goes up to 5.5°. Further, FIG. 3 along with the table 1 below illustrates the curved dimensions.

TABLE 1
curved dimensions of FIG. 3

| Size | Dim A | Dim B | Dim C | R1 | R2 |
|---|---|---|---|---|---|
| A | 1.8 | 13 | 6.6 | 17.60 | 10 |
| B | 1.2 | 13.83 | 5 | 19 | 12 |
| C | 1.2 | 14 | 4.3 | 19 | 12 |
| D | 1.3 | 15 | 4 | 21 | 12 |
| E | 1.3 | 15.5 | 4 | 21 | 13 |
| F | 0.5 | 16.70 | 4 | 25 | 16.07 |
| G | 1 | 17.70 | 3.9 | 25 | 19 |
| H | 0.3 | 17.70 | 3.9 | 26 | 19 |

After that curve, plans are created at 0°, 15°, 30°, 45°, 60°, 75° and 90° rotation of femur with respect to tibial insert 104 and sketches are added. Dimension for section is as follows. 4° angle is constant for all sections. Using all sketches at different position curved part is added. Further, FIG. 4A, and FIG. 4B along with the table 2 below illustrates the curved dimensions.

TABLE 2
Dimensions of loft profile and section of loft profile

| Size | Dim A | Dim B |
|---|---|---|
| A | 4 | 1 |
| B | 4.29 | 0.5 |
| C | 4.29 | 0.5 |
| D | 4 | 0.5 |
| E | 4 | 0.5 |
| F | 7 | 0.5 |
| G | 7 | 1 |
| H | 7 | 1 |

For progressive angle, tibial insert 104 some changes created in revision tibial insert 104. Tibial post 112 with is increased at medio-lateral and anterior-posterior direction to maintain the I-E rotation 0.4° up-to 5.5°. Draft 4° is added on tibial post 112 from bottom surface. Further, FIG. 5A, FIG. 5B, FIG. 5C and FIG. 5D illustrate various views of tibial insert 104. Further FIG. 5D illustrates Medio lateral Post distances (Dim A) of tibial insert 104 in reference to the below table.

| Size | Medio lateral distances (Dim A) |
|---|---|
| A-12 | 14.90 |
| BC-12 | 17.60 |
| BC-34 | 17.60 |
| BC-56 | 17.60 |
| DE-34 | 18.70 |
| DE-56 | 18.70 |
| F-4 | 22.1 |
| FGH-56 | 22.1 |
| FGH-7 | 22.1 |

Femur and insert rotate internally and externally there are different contact point with different angle. When femur and insert do not have any I-E rotation the three-contact point lies on same line and when it rotates internal-external their contact point should be diagonal. FIG. 6A FIG. 6B and FIG. 6C illustrates Femur insert assembly (after I-E rotation of the femur on the insert contact point are marked).

Although the description provides implementations of a revision femur prosthesis, it is to be understood that the above descriptions are not necessarily limited to the specific features or methods. Rather, the specific features and methods are disclosed as examples of implementations for a revision femur prosthesis.

1. A femur prosthesis comprising:
a femoral component (102), wherein the femoral component (102) is fabricated from (Cobalt-Chromium-Molybdenum (CoCrMo Alloy), with the femoral component (102) is configured for attachment to a femur via a steam base 106, where in the femoral component (102) comprises a 3d curved profile (114) in its box (202); and
a tibial insert (104), wherein the tibial insert (104) is mounted on the femoral component (102), wherein the tibial insert (104) made of Ultra-High Molecular Weight Polyethylene (UHMWPE) material, wherein the tibial insert (104) comprises an articulating surface (508), wherein the articulating surface (508) interfaces with the femoral component (102) to facilitate flexion-extension and internal-external rotational movement, wherein the tibial insert (104) comprises a tibial post (112), wherein the tibial post (112) comprises an increased width in medio-l lateral and anterior-posterior directions, wherein based on the 3d curved profile in the box (202), an internal-external rotation progressively increases for different flexion angles of the femoral component (102), achieving an internal-external angle ranging between 0.4° to 5.5°.

2. The femur prosthesis as claimed in claim 1, wherein the femoral component (102) has configurations in both Left and Right orientations.

3. The femur prosthesis as claimed in claim 1, wherein the femoral component (102) has a provision for distal and posterior augment attachment.

4. The femur prosthesis as claimed in claim 1, wherein the tibial post (112) includes a 4° draft added from a bottom surface of the tibial post (112).

5. The femur prosthesis as claimed in claim 1, wherein the tibial insert (104) has a 7° posterior slope on the articulating surface (508).

6. The femur prosthesis as claimed in claim 1, wherein the femoral component (102) comprises a deeper patella groove.

7. The femur prosthesis as claimed in claim 1, wherein the tibial insert (104) comprises chamfers at its anterior and posterior sides.

8. The femur prosthesis as claimed in claim 7, wherein the anterior chamfer facilitates easy patella movement, and the posterior chamfer provides space for ligaments.

9. The femur prosthesis as claimed in claim 1, wherein at a 0° flexion angle, the internal-external rotation is up to 1° due to clearances in the box (202) and tibial post (112).

10. The femur prosthesis as claimed in claim 1, wherein for a 90° flexion angle, the internal-external rotation ranges up to 5.5° to 6°.
